(19) United States
(12) Patent Application Publication
(10) Pub. No.: US 2023/0218697 A1
(43) Pub. Date: Jul. 13, 2023

(54) COMPOSITION COMPRISING FIBRE AND MULBERRY

(71) Applicant: SOCIETE DES PRODUITS NESTLE S.A., Vevey (CH)

(72) Inventors: ZHIZHONG DONG, Beijing (CN); YUJIE SHI, Beijing (CN)

(21) Appl. No.: 17/996,649

(22) PCT Filed: Apr. 23, 2021
(86) PCT No.: PCT/EP2021/060663

§ 371 Date: Oct. 20, 2022

(30) Foreign Application Priority Data
Apr. 24, 2020 (WO) PCT/CN2020/086705

(51) Int. Cl. A61K 36/605; A61K 47/36; A61P 3/10

(52) U.S. Cl. CPC A61K 36/605; A61K 47/36; A61P 3/10

(57) ABSTRACT

A combined preparation of fibre and a mulberry component is disclosed, as well as a composition comprising the combined preparation as an active components, their use and methods of using them, for controlling blood glucose response, enhancing weight loss and/or reducing fat mass gain. The fibre and mulberry component mutually potentiate or synergistically interact to realize the above uses.

### TECHNICAL FIELD

The invention relates to a composition comprising fibre and mulberry, the use of said composition and a method of using the same. In particular, the invention relates to a combined composition comprising fibre and a mulberry component as the only active ingredients, which mutually potentiate or synergistically interact for the maintenance of glycaemia, the management of blood glucose, including both fasting glycaemia and postprandial glycaemia, for promoting weight loss, reducing fat mass gain and/or preventing or treating obesity.

### BACKGROUND OF THE INVENTION

Obesity and associated metabolic disorders, including hyperlipidaemia and hyperglycaemia, are big concerns worldwide, especially for the middle-aged to senior people. Long-term hyperglycaemia will cause damage to various tissues and organs of the body and result in acute and/or chronic complications, such as dehydration, electrolyte disturbances, nutrition deficiency, impaired renal function, lesion to nerves or fundus oculi, cardiovascular and cerebrovascular diseases, diabetic foot and the like.

During the past decades, the prevalence of obesity has increased worldwide to epidemic proportion. Approximately 1 billion of people worldwide are overweight or obese, conditions that increase mortality, mobility and economical costs. Obesity develops when energy intake is greater than energy expenditure, the excess energy being stored mainly as fat in adipose tissue. Body weight loss and prevention of weight gain can be achieved by reducing energy intake or bioavailability, increasing energy expenditure and/or reducing storage as fat. Obesity represents a serious threat to health because it is associated with an array of chronic diseases, including diabetes, atherosclerosis, degenerative disorders, airway diseases and some cancers.

Diabetes mellitus, or commonly referred to as diabetes, is a carbohydrate metabolism disorder resulting from insufficient production of, or reduced sensitivity to, insulin. Diabetes is often characterized as Type 1 or Type 2. The main manifestation of Type 2-diabetes includes excessive blood glucose levels following a meal due to inadequate first phase insulin secretion, i.e., excessive postprandial glycaemia levels. In the individuals suffering from Type 2-diabetes, the response to increased blood glucose levels and the modulation of such levels that would otherwise occur in a healthy individual is reduced or absent and thus results in an excessive spike in postprandial glycaemia levels. This is particularly significant given the well-established correlation between effective blood glucose control in a diabetic subject and the risk of developing cardiovascular or circulatory diseases or disorders, especially microvascular and macrovascular complications from such diseases or disorders. As such, controlling postprandial glycaemia levels in a diabetic individual is an important step in reducing the development of cardiovascular or circulatory diseases, and of course the subsequent development of cardiovascular related conditions such as retinopathy, neuropathy, nephropathy, and so forth.

Fasting glycaemia is also one of the common indexes in diabetes diagnosis, which reflects the function of β-cells of Langerhans islets and represents the excretion function of basal insulin. Generally, impaired fasting glycaemia, with a value of 6.1 to 7.0 mmol/l represents a transition stage from normal condition to diabetes, and appropriate diet and sports physical therapy or in combination with hypoglycaemic agents may normalize the blood glucose so as to prevent the development of diabetes. Therefore, keeping the fasting glycaemia well managed has great benefit in preventing diabetes.

Hence, the maintenance of glycaemia or the management of blood glucose, including both fasting glycaemia and postprandial glycaemia, is of great importance in preventing or treating diabetes and the relevant diseases, such as microvascular and macrovascular disorders, cerebrovascular diseases, circulatory disorders, and related complications including retinopathy, neuropathy, nephropathy and so forth.

There are many ways in which a subject can attempt to effectively control blood glucose levels, including both the fasting glycaemia and the postprandial glycaemia that so commonly occur in diabetes. Such methods often include dietary and lifestyle changes as well as the use of insulin and/or other medications designed to ultimately control blood glucose levels. Many individuals, however, require the use of injectable insulin and/or the administration of oral medications, all of which can be costly and in some cases result in undesirable secondary effects.

There have been various efforts in the art to develop pharmaceutical or nutritional compositions showing beneficial effects on blood glucose management or maintenance of glycaemia. For example, CN104473152A discloses a biological medicated food equivalent to an acarbose physical method for reducing postprandial glycaemia. In said application, at least five components of barley glucan, mulberry leaf, Chinese yam, Konjaku and oat powder are provided and the advantageous of all raw materials are fully played to produce the claimed effects. WO2013/078658 reports effects of mulberry juice on blood glucose levels following consumption of high glycaemic index carbohydrate. EP3145332 A1 reports reduction of post-meal glucose and insulin spikes following administration of a composition comprising a combination of white kidney bean extract, white mulberry extract and green coffee extract administered 15 minutes before a carbohydrate meal.

There is therefore a need for a composition or method that would complement standard treatment of diabetes mellitus, or reduce the reliance upon the use of insulin or other medications, or provide useful options to effectively control blood glucose levels, including both the fasting glycaemia and the postprandial glycaemia. There is also a need for a composition or method that can support weight management.

There is also a need for a nutritional composition that would assist in reducing the reliance upon the use of insulin or other medications, or in providing useful options to effectively assist in controlling blood glucose levels, including both the fasting glycaemia and postprandial glycaemia. There is also a need for a nutritional composition that can assist in enhancing weight loss and/or reducing fat mass gain, and assist in the prevention or treatment of obesity.

It is therefore an object of the invention to provide a new combination or composition with desired control on blood glucose levels, including both fasting glycaemia and postprandial glycaemia, that can promote weight loss and/or reduce fat mass gain, or to at least provide a useful alternative.

### SUMMARY OF THE INVENTION

The inventors have surprisingly found that insulin sensitivity and glucose tolerance can be improved, specifically blood glucose levels can be controlled and regulated, weight loss can be enhanced and fat mass gain reduced, by administering to a subject in need thereof a combined preparation of a fibre, and a mulberry component. When used in combination, the fibre and mulberry mutually potentiate or synergistically interact to control and regulate blood glucose levels, enhance weight loss and/or reduce fat mass gain in a subject, to an extent greater as compared with the fibre or mulberry alone. The combination or composition may contain lower amounts of the fibre or mulberry than required when using either alone. The mulberry component may be a mulberry plant material, a mulberry extract, or a combination thereof. In preferred embodiments the mulberry component is a mulberry leaf extract (MLE). The fibre is preferably a soluble fibre. The soluble fibre may be a selected from the group consisting of FOS, GOS, inulin, β-glucan, resistant maltodextrins, acacia gum, partially hydrolysed guar gum (PHGG), polydextrose and combinations thereof. In a preferred embodiment the fibre comprises PHGG. In an embodiment the fibre is selected from the group consisting of FOS, inulin, acacia gum, PHGG or a combination thereof. In a specific embodiment the fibre is a combination of FOS, inulin, acacia gum and PHGG.

In a first aspect of the invention, there is provided a combined preparation of fibre and a mulberry component, wherein the mulberry component is a mulberry plant material, a mulberry extract, or a combination thereof.

In a second aspect of the invention, there is provided a combined preparation of fibre and a mulberry component, wherein the mulberry component is a mulberry plant material, a mulberry extract, or a combination thereof, for the maintenance of glycaemia, the management of blood glucose, including both fasting glycaemia and postprandial glycaemia, for enhancing weight loss and/or reducing fat mass gain.

In a third aspect of the invention, there is provided a composition comprising fibre and a mulberry component, for the maintenance of glycaemia, the management of blood glucose, including both fasting glycaemia and postprandial glycaemia, for enhancing weight loss and/or reducing fat mass gain, wherein the mulberry component is a mulberry plant material, a mulberry extract, or a combination thereof.

In a fourth aspect of the invention, there is provided a composition comprising fibre and a mulberry component that would assist in controlling blood glucose levels, including both the fasting glycaemia and postprandial glycaemia, assist promoting weight loss and/reducing fat mass gain, and assist in the treatment or prevention of obesity.

In a fifth aspect of the invention, there is provided a use of the combined preparation or the composition as described above for the manufacture of medicaments for the maintenance of glycaemia, the management of blood glucose, including both fasting glycaemia and postprandial glycaemia , for enhancing weight loss and/or reducing fat mass gain.

In a six aspect of the invention, there is provided a use of the composition as described above for the manufacture of health products to assist in controlling blood glucose levels, including both the fasting glycaemia and postprandial glycaemia, weight loss and/or reducing fat mass gain, and/or in the prevention or treatment of obesity.

In a seventh aspect of the invention, there is provided a use of fibre for the manufacture of a composition to be administrated together with a mulberry component, for the maintenance of glycaemia, the management of blood glucose, including both fasting glycaemia and postprandial glycaemia, for enhancing weight loss and/or reducing fat mass gain, wherein the mulberry component is a mulberry plant material, a mulberry extract, or a combination thereof.

In an eighth aspect of the invention, there is provided a use of a mulberry component for the manufacture of a composition to be administrated together with fibre for the maintenance of glycaemia, the management of blood glucose, including both fasting glycaemia and postprandial glycaemia, for enhancing weight loss and/or reducing fat mass gain, wherein the mulberry component is a mulberry plant material, a mulberry extract, or a combination thereof.

In a ninth aspect of the invention, there is provided a method for the maintenance of glycaemia or the management of blood glucose, including both fasting glycaemia and postprandial glycaemia, in a subject in need thereof, comprising administering a mutually potentiating amount of fibre together with a mutually potentiating amount of a mulberry component to said subject, preferably the amounts of both the components synergistically produce the technical effects greater than their additive effects, wherein the mulberry component is a mulberry plant material, a mulberry extract, or a combination thereof. There is also provided a method for enhancing weight loss and/or reducing fat mass gain in a subject in need thereof comprising administering a mutually potentiating amount of fibre together with a mutually potentiating amount of a mulberry component to said subject, to produce the technical effects greater than either component alone, wherein the mulberry component is a mulberry plant material, a mulberry extract, or a combination thereof.

In a preferred embodiment of each of the above aspects, the mulberry component is a mulberry leaf extract (MLE).

In specific embodiments of each of the above aspects, fibre and a mulberry component are used as the only active ingredients, and/or are administered orally.

Accordingly, the combined preparation, composition, uses and methods of the present invention offer a natural therapeutic or nutritional option that may contribute to the maintenance of optimal glycaemic control in subjects that are pre-diabetic, have impaired glucose tolerance, impaired fasting glycaemia, or have type-2 diabetes or related complications including retinopathy, neuropathy, nephropathy and the like. These benefits are also advantageously achieved in such individuals without experiencing many of the complications often associated with the administration of oral anti-diabetic medications. The combined preparation, compositions, uses and methods of the present invention also offer an effective means support weight management.

### BRIEF DESCRIPTION OF THE FIGURES

In the figures, MLE refers to mulberry leaf extract, as described in the examples.

FIG. 1 shows fasting serum insulin concentrations in male C57BL/6 mice. Values are means±S.E.M.; n=10., **p<0.01 vs relative control.

FIG. 2 shows fasting serum glucose concentrations in male C57BL/6 mice. Values are means±S.E.M.; n=10., **p<0.01 vs relative control.

FIG. 3 shows blood glucose AUC in male C57BL/6 mice following OGTT. Values are means±S.E.M.; n=10., **p<0.01 vs relative control.

FIG. 4 shows (A) (B) final body weight, (C) (D) body weight gain in male C57BL/6 mice after four weeks treatment. Values are means±S.E.M.; n=10. *p<0.05, **p<0.01.

FIG. 5 shows (A) (B) epididymal fat mass and (C) (D) perirenal fat mass in male C57BL/6 mice after four months treatment. Values are means±S.E.M.; n=10, *p<0.05, **p<0.01.

### DETAILED DESCRIPTION

In a first aspect of the invention, there is provided a combined preparation of fibre and a mulberry component, wherein the mulberry component is a mulberry plant material, a mulberry extract, or a combination thereof. The fibre is preferably a soluble fibre. The fibre may be FOS, GOS, inulin, β-glucan, resistant maltodextrin, partially hydrolysed guar gum (PHGG), polydextrose or any combination thereof. In a preferred embodiment the fibre comprises PHGG. In an embodiment the fibre is selected from the group consisting of FOS, inulin, acacia gum, PHGG or a combination thereof.

In a further embodiment of the invention, the mulberry plant material can be of any Morus origin including but not limited to: White Mulberry (Morus alba L.), Black Mulberry (Morus nigra L.), American Mulberry (Morus celtidifolia Kunth), Red Mulberry (Morus rubra L.), hybrid forms between Morus alba and Morus rubra, Korean Mulberry (Morus australis), Himalayan Mulberry (Morus laevigata), and combinations thereof.

In a further embodiment of the invention, the mulberry plant material is derived from different parts of mulberry tree, including barks (trunk, twig or root), roots, buds, twigs, young shoots, leaves, and fruits.

In a further embodiment of the invention, the mulberry materials can be in the form of extracts or dried powders such as dried powders milled from different parts of a mulberry tree.

In a further embodiment of the invention, the extracts of mulberry plant material (herein referred to as “mulberry extracts”) can be derived from the whole or any part of the plant, such as barks (trunk, twig or root), roots, buds, twigs, young shoots, leaves, fruits or a combination thereof. The starting materials may be fresh, frozen or dried material. After filtration or dialysis, the extracts may be used as a liquid or dried solid.

In a still further embodiment of the invention, the mulberry plant materials or extracts thereof are mulberry leaves extracts or a milled powder of mulberry leaves. In a preferred embodiment the mulberry component is a mulberry leaf extract.

In a further embodiment of the invention, the mulberry extracts are prepared by known procedures in the art, e.g., by extracting the mulberry materials, including the whole or part of the mulberry plant, with solvent such as alcohol (e.g., n-butanol, ethanol or the like) and/or water.

In a further embodiment of the invention, the weight ratio of fibre to mulberry component may range from about 1:10 to about 40:1, preferably from about 1:1 to about 30:1, more preferably from about 4:1 to about 25:1, for example about 4:1 to about 20:1, for example from about 5:1 to about 10:1, or for example about 25:1.

In a further embodiment of the invention, the combined preparation is in the form of powder.

In a second aspect of the invention, there is provided a combined preparation as defined above for the maintenance of glycaemia, the management of blood glucose, including both fasting glycaemia and postprandial glycaemia, for enhancing liver detoxification and/or for reducing liver oxidative damage.

In a further embodiment, the combined preparation is useful in for preventing and/or treating hyperglycaemia and/or the relevant conditions, such as prediabetes, diabetes and especially type-2 diabetes, impaired glucose tolerance, diabetic retinopathy, diabetic neuropathy, diabetic nephropathy, and/or for preventing or treating obesity, so can be used to manufacture medicaments for treating and/or preventing the above conditions

In a third aspect of the invention, there is provided a composition comprising fibre and a mulberry component, for the maintenance of glycaemia, the management of blood glucose, including both fasting glycaemia and postprandial glycaemia, for enhancing weight loss and/or reducing fat mass gain, wherein the mulberry component is a mulberry plant material, a mulberry extract, or a combination thereof.

In a fourth aspect of the invention, there is provided a composition comprising fibre and a mulberry component that would assist in controlling blood glucose levels, assist in enhancing weight loss and/or reducing fat mass gain, and assist in preventing or treating obesity, so can be used to manufacture nutrients for the above purposes, wherein the mulberry component is a mulberry plant material, a mulberry extract, or a combination thereof.

In further embodiments of the third and fourth aspects of the invention, all the specific definitions for fibre and a mulberry component, as described above for the combined preparation, also apply to the composition.

In a still further embodiment of the invention, the composition is useful for preventing and/or treating hyperglycaemia and/or the relevant conditions thereof, such as prediabetes, diabetes and especially type-2 diabetes, impaired glucose tolerance, diabetic retinopathy, diabetic neuropathy, diabetic nephropathy, and/or preventing or treating obesity.

In a further embodiment of the invention, the composition is for oral administration.

In a further embodiment of the invention, the compositions most typically comprise from about 5% to about 95%, including from about 10% to about 95%, also including from about 50% to about 90%, also including from about 70% to about 90%, and also including from about 80% to about 90%, fibre by dry weight of the compositions.

In a further embodiment of the invention, the compositions may typically comprise from about 1% to about 50%, including from about 2% to about 30%, such as from about 5% to about 20%, and also including from about 10% to about 15% of mulberry component by dry weight of the composition. In other embodiments the compositions may comprise comprise from about 1% to about 10%, including from about 1% to about 5%, also including from about 1% to about 4%, and also including from about 1% to about 2% of a mulberry component by weight of the composition. The mulberry component is a mulberry plant material, a mulberry extract, or a combination thereof.

In a further embodiment of the invention, the composition further comprises a filler material, a fat source, a protein source, a flowing agent, a stabilizer, a preservative, an anti-oxidant, an acid, a buffer, a sweetener, an intense sweetener, a colorant, a flavour, a flavour enhancer, an emulsifying agent, an anti-caking agent, a lubricant, or any combination thereof.

In a fourth aspect of the invention, there is provided a use of the combined preparation as defined above for the maintenance of glycaemia, the management of blood glucose, including both fasting glycaemia and postprandial glycaemia, for enhancing liver detoxification and/or for reducing liver oxidative damage, e.g., for preventing and/or treating hyperglycaemia and/or the relevant conditions, such as prediabetes, diabetes and especially type-2 diabetes, impaired glucose tolerance, diabetic retinopathy, diabetic neuropathy, diabetic nephropathy, and/or for the treatment or prevention of obesity.

In a fifth aspect of the invention, there is provided a use of fibre for the manufacture of a composition to be administrated in combination with a mulberry component, wherein the mulberry component is a mulberry plant material, a mulberry extract, or a combination thereof, for the maintenance of glycaemia, the management of blood glucose, including both fasting glycaemia and postprandial glycaemia, for promoting weight loss and/or reducing fat mass gain, e.g., for preventing and/or treating hyperglycaemia and/or the relevant conditions, such as prediabetes, diabetes and especially type-2 diabetes, impaired glucose tolerance, diabetic retinopathy, diabetic neuropathy, diabetic nephropathy, and/or preventing or treating obesity.

In a sixth aspect of the invention, there is provided a use of a mulberry component, wherein the mulberry component is a mulberry plant material, a mulberry extract, or a combination thereof, for the manufacture of a composition to be in combination with a fibre for the maintenance of glycaemia, the management of blood glucose, including both fasting glycaemia and postprandial glycaemia, for promoting weight loss and/or for reducing fat mass gain, e.g., for preventing and/or treating hyperglycaemia and/or the relevant conditions, such as prediabetes, diabetes and especially type-2 diabetes, impaired glucose tolerance, diabetic retinopathy, diabetic neuropathy, diabetic nephropathy, and/or for treating or preventing obesity.

In a seventh aspect of the invention, there is provided a method for the maintenance of glycaemia, the management of blood glucose, including both fasting glycaemia and postprandial glycaemia, for promoting weight loss and/or for reducing fat mass gain, e.g., for preventing and/or treating hyperglycaemia and/or the relevant conditions, such as prediabetes, diabetes and especially type-2 diabetes, impaired glucose tolerance, diabetic retinopathy, diabetic neuropathy, diabetic nephropathy, and/or for treating or preventing obesity in a subject in need thereof, comprising administering a mutually potentiating amount of a fibre together with a mutually potentiating amount of a mulberry component, wherein the mulberry component is a mulberry plant material, a mulberry extract, or a combination thereof, to said subject, preferably the amounts of both the components synergistically produce the technical effects greater than their additive effects.

In further embodiments for each of above aspects of the invention, fibre and mulberry component, wherein the mulberry component is a mulberry plant material, a mulberry extract, or a combination thereof, are used as the only active ingredients.

It is to be appreciated those certain features of the invention, which are, for clarity, described above and below in the context of separate embodiments, may also be provided in combination in a single embodiment. Conversely, various features of the invention that are, for brevity, described in the context of a single embodiment, may also be provided separately or in any sub-combination.

### Definitions

As used herein, the term “combined preparation” means that the combination partners (a) and (b) can be dosed independently or by different fixed combinations with distinguished amounts of the combination partners (a) and (b), i.e., simultaneously, sequentially or separately. The combination partners can then, e.g., be administered simultaneously or chronologically staggered, that is at different time points and with equal or different time intervals for any combination partner. Combination partners (a) and (b) can be contained in the same composition, with vehicles, excipients or other ingredients useful for the proposed applications; or they may be administered or applied in separate compositions as well as through different administration routes, in order to obtain a combined therapeutic effect. Preferably, combination partners (a) and (b) are contained in the same composition.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “mulberry plant materials” means any whole plant or any part thereof from Morus plants.

“Body mass index ” or “BMI” means the ratio of weight in Kg divided by the height in metres, squared.

“Obesity” is a condition in which the natural energy reserve, stored in the fatty tissue of animals, in particular humans and other mammals, is increased to a point where it is associated with certain health conditions or increased mortality. “Obese” is defined for an adult human as having a BMI greater than 30.

“Weight loss” in the context of the present invention is a reduction of the total body weight. Weight loss may for example refer to the loss of total body mass in an effort to improve fitness, health, and/or appearance.

“Weight management” or “weight maintenance” relates to maintaining a total body weight. For example, weight management may relate to maintaining a BMI in the area of 18.5-25 which is considered to be normal

As used herein, the term “potentiating” or “potentiation” means an increase of a corresponding pharmacological activity or therapeutical effect, respectively. Potentiation of one component of the combination according to the present invention by co-administration of another component according to the present invention means that an effect is being achieved that is greater than that achieved with either component alone.

As used herein, the term “synergistic” or “synergistically” means that the components, when taken together, produce a total joint effect that is greater than the sum of the effects of each component when taken alone.

As used herein, the words “comprises”, “comprising”, and similar words, are not to be interpreted in an exclusive or exhaustive sense. In other words, they are intended to mean “including, but not limited to.

As used herein, the term “and/or” used in the context of the “X and/or Y” should be interpreted as “X”, or “Y”, or “X and Y”.

As used herein, the term “about” when referring to a measurable value such as an amount is meant to encompass variations of ±20% or in some instances ±10%, or in some instances ±5%, or in some instances ±1%, or in some instances ±0.1% from the specified value, as such variations are appropriate to the disclosed composition.

Any percentages and ratios as used herein are by weight of the total preparation or composition, unless otherwise specified. All such weights as they pertain to listed ingredients are based on the active level and, therefore, do not include solvents or by-products that may be included in commercially available materials, unless otherwise specified.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 1 to 8, from 3 to 7, from 4 to 9, from 3.6 to 4.6, from 3.5 to 9.9, and so forth.

Any reference to singular characteristic or limitation of the present invention shall include the corresponding plural characteristics or limitations, and vice versa, unless otherwise specified or clearly implied to the contrary by the context in which the reference is made.

The combination, composition and method of the present invention may comprise, consist of, or consist essentially of the elements and features of the invention described herein, as well as any additional or optional ingredients, components, or features described herein or otherwise useful in a pharmaceutical or nutritional application.

Any reference to prior art documents in this specification is not to be considered an admission that such prior art is widely known or forms part of the common general knowledge in the field.

Unless defined otherwise, all technical and scientific terms, terms of art, and acronyms used herein have the meanings commonly understood by one of ordinary skill in the art in the field(s) of the invention, or in the field(s) where the term is used.

### Fibre

The combined preparation and the compositions of the present invention comprise fibre. Any dietary fibre, including soluble and insoluble fibre, that is known or otherwise suitable for use in an oral product is also suitable for use herein, provided that such a source is also compatible with, or is otherwise rendered to be compatible with, the other selected ingredients in the composition. With the proviso that the fibre is not beta-glucan.

Preferably one or more soluble fibers are used.

Beneficial effects of fibres on glucose response have been widely reported. Non-limiting examples of suitable soluble fibres include FOS, GOS, inulin, resistant maltodextrins, partially hydrolysed guar gum, polydextrose and combinations thereof.

Advantageously the fibre can also act as a filler material to augment the bulk properties of the composition.

Fibers, in combination with the Mulberry materials or extracts thereof described herein, may mutually potentiate or preferably act synergistically to control and regulate blood glucose levels such that glucose absorption into the bloodstream is retarded, thus lengthening the amount of time it takes a given amount of glucose to enter the bloodstream.

In an embodiment the fibre comprises PHGG. In an embodiment the fibre is a combination of FOS, inulin and acacia gum. In a specific embodiment the fibre is a combination of FOS, inulin, acacia gum and PHGG.

In another embodiment the fibre is selected from a polydextrose or a digestion resistant maltodextrin (such as Fibersol 2™ (Archer Daniels Midland Company)). In an embodiment the composition comprises soluble fibre and mulberry extract in a ratio fibre:mulberry extract of from about 1:1 to about 30:1, preferably from about 4:1 to about 10:1.

In another embodiment the fibre is a combination of FOS, inulin and acacia gum, optionally further in combination with PHGG and the composition comprises the fibre and mulberry extract in a ratio fibre:mulberry extract of from about 4:1 to about 30:1, such as about 25:1.

### Mulberry Component

The present invention involves using of a mulberry component. The mulberry component may be a mulberry plant material, a mulberry extract, or a combination thereof. Mulberry plant materials have been used centuries in traditional Chinese medicine as a common agent to treat a variety of conditions including diabetes, atherosclerosis, cancer as well as for boosting the immune system through potent antioxidant activity. Research has shown that mulberry plant materials comprise various physiologically active components including flavonoids, polyphenols, polysaccharides, 1-deoxynojirimycin (DNJ) identified as α-glucosidase inhibitor, fagomine, GABA or the like. So it can be useful in preventing or treating cardiovascular diseases, hyperlipidemia, diabetes, obesity and anti-aging.

Any source of the mulberry component that is known or otherwise suitable for human use is also suitable for use herein, provided that such a source is also compatible with, or is otherwise rendered to be compatible with, the other selected ingredients in the composition. The mulberry component may be a mulberry plant material, a mulberry extract, or a combination thereof.

The mulberry component applicable to the present invention can be of any Morus origin, including, but not limited to, White Mulberry (Morus alba L.), Black Mulberry (Morus nigra L.), American Mulberry (Morus celtidifolia Kunth), Red Mulberry (Morus rubra L.), hybrid forms between Morus alba and Morus rubra, Korean Mulberry (Morus australis), Himalayan Mulberry (Morus laevigata), and combinations thereof.

The mulberry component applicable to the present invention can be derived from different parts of mulberry tree, including barks (trunk, twig or root), roots, buds, twigs, young shoots, leaves, fruits or a combination thereof. The mulberry materials can be in the form of e.g. dried powders such as dried powders milled from different parts of the tree. The starting plant material of mulberry extracts can be fresh, frozen or dried mulberry materials. The extract may be used as a liquid or dried concentrated solid. Typically, such an extract includes from at least about 1% w/v 1-DNJ.

Preferably, the mulberry component used in the present invention are mulberry leaves or mulberry leaves extracts (MLE in the figures).

Mulberry extracts can be prepared by known procedures in the art, for example by extracting dried mulberry leave powders with water or 70% to 80% aqueous ethanol at 60° C. for 2 h, the extracts are then enriched through ion-exchange resin eluting with 0.25 mol·L⁻¹ aqueous ammonia, and the eluant is further extracted with n-butanol, concentrated and dried to provide the mulberry leaf extracts. References in this aspect can be made to Chao Liu et al., Comparative analysis of 1-deoxynojirimycin contribution degree to α-glucosidase inhibitory activity and physiological distribution in Morus alba L, Industrial Crops and Products, 70 (2015) p 309-315; Wenyu Yang et al., Studies on the methods of analyzing and extracting total alkaloids in mulberry, Lishizhen Medicine and Materia Medical Research, 2008(5); and CN104666427.

The mulberry components applicable to the present invention, for instance mulberry leaf extract, can also be commercially available, such as those from Naturex Ltd, Karallief Inc, USA, ET-Chem, China, Nanjing NutriHerb BioTech Co., Ltd, China, or from Phynova Group Ltd..

The compositions of the present invention may comprise an amount of mulberry components sufficient to potentiate or preferably synergistically interact with the β-glucan component of the composition in providing the desired effects. The compositions most typically, however, comprise from about 0.5% to about 50%, including from about 2% to about 30%, such as from about 5% to about 20%, and also including from about 10% to about 15% of mulberry component by weight of the composition. In other embodiments the compositions may comprise from about 1% to about 10%, including from about 1% to about 5%, also including from about 1% to about 4%, and also including from about 1% to about 2% of a mulberry component by weight of the composition. The mulberry component is a mulberry plant material, a mulberry extract, or a combination thereof.

In an embodiment of the present invention the composition comprises at least 1 mg 1-deoxynojirimycin (DNJ) per g dry weight, preferably at least 1 mg DNJ per g dry weight of the composition, preferably at least 2 mg DNJ, preferably at least 3 mg DNJ, preferably at least 4 mg DNJ, more preferably at least 5 mg DNJ, such as at least 6 mg DNJ per g dry weight of the composition. In one embodiment of the present invention the composition comprises about 1 to about 20 mg DNJ per g dry weight of the composition, preferably about 2 mg to about 10 mg DNJ per g dry weight of the composition, In one embodiment of the present invention the composition comprises about 5 to about 10 mg DNJ per g dry weight of the composition, preferably about 5 mg to about 8 mg DNJ per g dry weight of the composition, such as about 6 mg to about 7 mg DNJ per g dry weight of the composition.

In an embodiment, the composition is administered to an individual in a serving that provides at least about 1 mg DNJ, preferably at least about 5 mg DNJ per serving, more preferably at least about 10 mg DNJ per serving. In some embodiments, up to 50 mg DNJ are administered per serving of the composition. In some embodiments, up to 100 mg DNJ may be administered per serving of the composition.

The weight ratio of mulberry components to fibre in the present compositions or combined preparation may range from 1:40 to 1:1, preferably 1:30 to 1:1, including 1:10 to 1:4.

### Combined Preparation

For the present invention, the “combined preparation” means that combination partner (a) fibre and combination partner (b) mulberry component can be dosed independently or by different fixed combinations with distinguished amounts of (a) and (b), fibre and mulberry component can then, e.g., be administered simultaneously or chronologically staggered, that is at different time points and with equal or different time intervals for either of them. Fibre and mulberry component can be contained in the same composition, with vehicles, excipients or other ingredients useful for the proposed applications; or they may be administered or applied in separate compositions as well as through different administration routes, in order to obtain a combined therapeutic effect.

Simultaneous administration may, e.g., take place in the form of one fixed combination of (a) and (b), or by simultaneously administering (a) and (b) that are formulated independently.

Sequential use (administration) preferably means administration of one component of the combination at one time point, the other component at a different time point, that is, in a chronically staggered manner, preferably such that the combination shows more efficiency than the single component administered independently (especially showing potentiation or synergism).

Separate use (administration) preferably means administration of the components of the combination independently of each other at different time points, preferably meaning that the components (a) and (b) are administered such that no overlap of measurable blood levels of both compounds are present in an overlapping manner (at the same time).

Also combinations of two or more of sequential, separate and simultaneous administration are possible, preferably such that the combination components show a therapeutic effect that exceeds the effect found when the combination components are used independently at time intervals so large that no mutual effect on their therapeutic efficiency can be found.

The ratio of the total amounts of the combination partner (a) fibre to the combination partner (b) mulberry component to be administered in the combined preparation can be varied, e.g., in order to cope with the needs of a patient sub-population to be treated or the needs of the single patient which different needs can be due to the particular disease, age, sex, body weight, etc. of the patients.

Preferably, there is at least one beneficial effect, e.g., a mutual enhancing of the effect of the combination partners (a) fibre and (b) mulberry component, in particular, a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (a) and (b), and very preferably a strong potentiation or synergism of the combination partners (a) and (b).

### Compositions

For the present invention, the “composition” means a composition containing the combination of fibre and mulberry component. The compositions of the present invention may include any optional additional ingredients, including conventional food additives (synthetic or natural), for example one or more of filler material, acidulants, thickeners, buffers or agents for pH adjustment, chelating agents, colorants, emulsifiers, excipient, flavor agent, mineral, osmotic agents, preservatives, stabilizers, texturizers, vitamins and the like. The optional ingredients can be added in any suitable amount.

Suitable compositions for the present invention may be in the form of powders, granules, tablets, chewables, soft gels, sachets, solutions (e.g. tonics), liquid suspensions, emulsions, or concentrate.

The compositions of the present invention may be administered by any means suitable for human or animal administration, in particular for administration in any part of the gastrointestinal tract. Enteral administration, oral administration, and administration through a tube or catheter are all covered by the present disclosure.

### Optional Ingredients

The combined preparation or the composition of the present invention may further comprise other optional agents that are useful for the maintenance of glycaemia, the management of blood glucose, for liver detoxification or for liver protection.

The combined preparation or the compositions may further comprise minerals suitable for use in a nutritional product. Non-limiting examples thereof include phosphorus, sodium, chloride, magnesium, manganese, iron, copper, zinc, iodine, calcium, potassium, chromium, chromium picolinate, molybdenum, selenium, and combinations thereof.

The combined preparation or the compositions may further comprise any vitamins or similar other materials suitable for human or animal use. Non limiting examples thereof include carotenoids (e.g., β-carotene, zeaxanthin, lutein, lycopene), biotin, choline, inositol, folic acid, pantothenic acid, vitamin A, thiamine (vitamin B1), riboflavin (vitamin B2), niacin (vitamin B3), pyridoxine (vitamin B6), cyanocobalamin (vitamin B12), ascorbic acid (vitamin C), vitamin D, vitamin E, vitamin K, and various salts, esters or other derivatives thereof, and any combinations thereof. Vitamin C, vitamin D and/or vitamin B12 are particularly useful in the composition. In a preferred embodiment the composition is supplemented with one or more vitamin and/or mineral selected from vitamin C, vitamin B3, zinc or any combination thereof.

### Use

The combined preparation according to the present invention is effective in reducing blood glucose.

Accordingly, the combined preparation, the composition, the use and method according to the present invention may be used for preventing and/or treating hyperglycemia and/or the relevant conditions thereof, such as prediabetes, diabetes and especially type-2 diabetes, impaired glucose tolerance, impaired insulin sensitivity, diabetic retinopathy, diabetic neuropathy, diabetic nephropathy.

The combined preparation, the composition, the use and method according to the present invention may be further used in weight management.

The combined preparation, the composition, the use and method according to the present invention may be further used in promoting weight loss and/or reducing fat mass gain.

The combined preparation, the composition, the use and method according to the present invention may be further used in the prevention or treatment of obesity.

The composition of the present invention may assist in the maintenance of glycaemia, the management of blood glucose, preferably assist in lowering blood glucose; promote weight loss, reduce fat mass gain, and may assist the prevention or treatment of obesity. It should be noted that embodiments and features described in the context of one of the aspects of the present invention also apply to the other aspects of the invention.

All patent and non-patent references cited in the present application, are hereby incorporated by reference in their entirety.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

The invention is further described with reference to the following examples. It will be appreciated that the invention as claimed is not intended to be limited in any way by these examples.

### EXAMPLES

The practice of the present invention will employ, unless otherwise indicated, conventional techniques which are known and available to a person skilled in the art.

By way of example and not limitation, the following Examples are illustrative of various embodiments of the present disclosure. The Examples are given solely for the purpose of illustration and are not to be construed as limitations, as many variations thereof are possible without departing from the spirit and scope of the disclosure. In all the examples, concentrations of components are given as w/w % based on the whole product formulation.

### Example 1

Materials Mulberry leaf extract for this study was purchased from Naturex Ltd. The extract was selective extract of mulberry leaf, containing 1% DNJ. Raw materials for Fiber B (FOS, Inulin, Acacia Gum+PHGG) and Fiber A (FOS, Inulin, Acacia Gum,) were purchased from from Taiyo. Ltd (Partially Hydrolyzed Guar Gum), Jebsen & Co. Ltd (Acacia gum), BENEO. Ltd (Inulin, Fructooligosaccharide).

Animals

Male 30-day old C57/BL6 mice weighed 20-25 g were purchased from a commercial breeder (SLAC, Shanghai) and were housed in SPF (specific parasite free) environment at 22° C. with a 12 hours light-dark cycle. All the mice were fed with chow diets for 7 days to allow adaptation to the environment and diet. The body overweight model was made by feeding rats with a high fat diet (HFD) (45% kcal fat content). The mice were randomly sub-divided into seven groups (10 mice per group): (i) control group (chow diet), (ii) HFD group (high fat diet (HFD), 45% kcal fat content as in Example 2), (iii) HFD with 200 mg/kg/day mulberry leaf extract (MLE), (iv) HFD with 5 g/kg/day Fiber A, (v) HFD with 5 g/kg/day Fiber B, (vi) HFD with MLE/Fiber A combination and (vii) HFD with MLE/Fiber B combination. The dose in combination for each ingredient is the same as the single ingredient group. The treatment was given at the same time with HF diet through gavage. The mice body weights and their food consumptions were recorded twice a week. The mice were sacrificed 4 months after treatments and HFD feed, and blood, liver, muscle and adipose tissues were collected and frozen at −75° C. In addition, the epididymal fat and perirenal fat were immediately excised and weighed. All of the procedures were performed in accordance with the United States Public Health Services Guide for the Care and Use of Laboratory Animals, and all efforts were made to minimize the suffering and the number of animals used in this study.

Oral Glucose Tolerance Test (OGTT)

OGTT was performed under conscious state before the treatment started and one week before the animal sacrifice to avoid the stress to animal body system. The OGTT (2.5 g maltose/kg body weight) was administrated following the previous published methods. All mice were fasted overnight before OGTT. Blood was taken from the retrobulbar vein at 0, 5, 15, 30, 60, 120 and 180 min after the oral maltose administration. Plasma glucose concentrations were determined by the glucose oxidase method.

Biochemical Assays for Blood

The levels of insulin, leptin, IL-1, IL-6, free fatty acid, triglyceride, cholesterol, HDL-cholesterol, and LDL-cholesterol in serum and the levels of free fatty acid in feces were assayed with routine clinical assays.

Statistical Analysis

Data are presented as means±S.E.M. Statistical significance was evaluated with one-way ANOVA followed by LSD post hoc analysis. In all comparisons, the level of significance was set at p<0.05.

Effects on Insulin Sensitivity

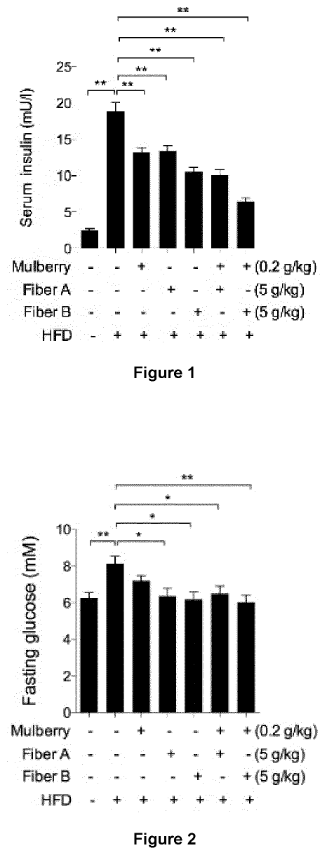

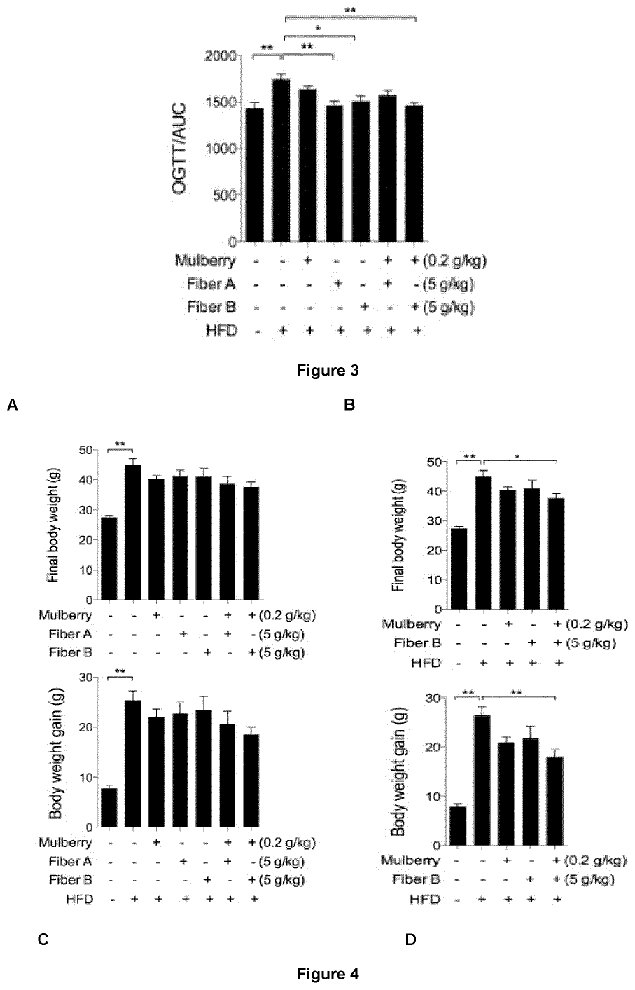

Mice serum was collected after the sacrifice. Serum glucose (A) and insulin (B) was measured. OGTT was performed one week before the mice sacrifice. Blood was taken from the retrobulbar vein at 0, 15, 30, 60, 120 and 180 min after the oral glucose administration. As seen in FIGS. 1, 2 and 3, HFD significantly increased mice fasting glucose, OGTT and insulin contents, which was significantly reduced by MLE and fibers (FIGS. 1-3, values are mean±SEM, N=10. **p<0.01 vs. relative control). The combination of MLE and fibers has better effects compared with single treatment (FIGS. 1-3). The combination of MLE and fiber B has synergistic effects compared with single treatment on insulin content (FIG. 1).

Effects on HFD Induced Body Weight

During four month HFD feeding and various supplements, mice body weight was measured twice a week. HFD significantly increase body weight and both MLE and fibers was found to successfully decrease the body weight (FIG. 4). The combination of MLE and fibers has better effects compared with single treatment. (FIG. 4). The most significant effects on decreasing body weight were observed with the combination of MLE and fibre B (FIGS. 4 B, D).

Effects on Epididymal Fat Mass and Perirenal Fat Mass

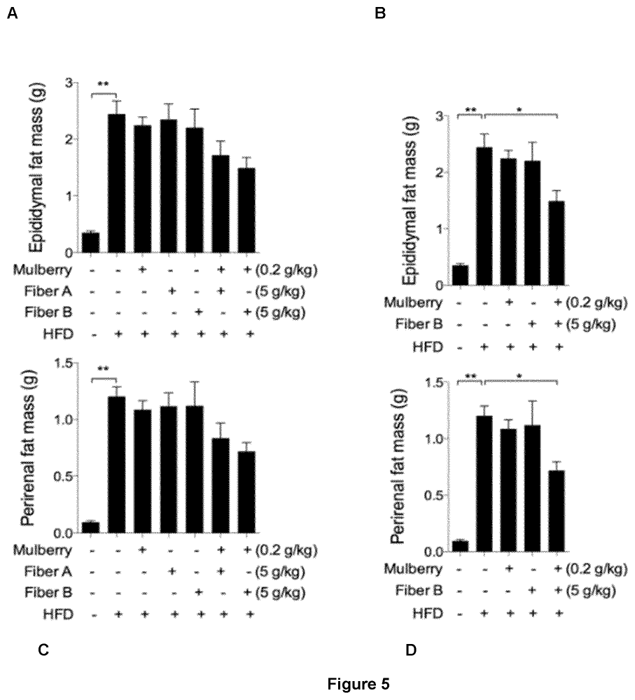

Similar with body weight gain result, both MLE and fibers were found to successfully decrease the Epididymal fat mass and perirenal fat mass. The combination of MLE and fibers has better effects compared with single treatment. (FIGS. 5 A, C). The combination of MLE and fiber B has synergistic effects compared with single treatment (FIGS. 5 B, D). Values are mean±SEM, N=10. *p<0.05, **p<0.01 vs. relative control.

1. A combined preparation of fibre and a mulberry component, wherein the mulberry component is selected from the group consisting of a mulberry plant material, a mulberry extract, and combinations thereof.

2. A method for the maintenance of glycemia, the management of blood glucose, for supporting weight loss and/or weight maintenance, comprising administering a composition comprising fibre and a mulberry component wherein the mulberry component is selected from the group consisting of a mulberry plant material, a mulberry extract, and combinations thereof.

3. The method according to claim 2, wherein the composition is for use in the prevention or treatment of obesity.

4. The method according to claim 2, wherein the mulberry component originates from White Mulberry (Morus alba L.), Black Mulberry (Morus nigra L.), American Mulberry (Morus celtidifolia Kunth), Red Mulberry (Morus rubra L.), hybrid forms between Morus alba and Morus rubra, Korean Mulberry (Morus australis), Himalayan Mulberry (Morus laevigata), and combinations thereof.

5. The method according to claim 2, wherein the mulberry plant material is selected from the group consisting of: whole mulberry, mulberry bark, mulberry root, mulberry bud, mulberry twig, mulberry shoot, mulberry leave, mulberry fruit, and combinations thereof.

6. The method according to claim 2, wherein said mulberry component is mulberry leaf extract.

7. The method according to claim 2, wherein the weight ratio of fibre to mulberry component ranges from 20:1 to 1:1.

8. The method according to claim 2, wherein the fibre is selected from the group consisting of FOS, GOS, inulin, β-glucan, resistant maltodextrins, acacia gum, partially hydrolysed guar gum (PHGG), polydextrose and combinations thereof.

9. The method according to claim 8, wherein the fibre comprises PHGG.

10. The method according to claim 2, wherein the composition is in a form selected from the group consisting of a powder, a paste, a liquid, and a tablet.

11. The method according to claim 2, wherein the composition comprises from about 5% to about 95% of the mulberry component by dry weight of the composition.

12. (canceled)

13. The method according to claim 2, for preventing and/or treating hyperglycemia and/or the relevant conditions selected from the group consisting of prediabetes, diabetes, impaired glucose tolerance, impaired fasting glycaemia, impaired insulin sensitivity, diabetic retinopathy, diabetic neuropathy, and diabetic nephropathy.

14. (canceled)

15. A method for the maintenance of glycaemia or the management of blood glucose, including both fasting glycaemia and postprandial glycaemia, for enhancing weight loss and/or reducing fat mass gain, comprising administering a combined preparation of fibre and a mulberry component, wherein the mulberry component is selected from the group consisting of a mulberry plant material, a mulberry extract, and combinations thereof to a subject in need thereof.

16. The method according to claim 15, for preventing and/or treating conditions selected from the group consisting of prediabetes, diabetes, impaired glucose tolerance, impaired fasting glycaemia, impaired insulin sensitivity, diabetic retinopathy, diabetic neuropathy, and diabetic nephropathy.
